(19) United States
(12) Patent Application Publication
(10) Pub. No.: US 2025/0292610 A1
(43) Pub. Date: Sep. 18, 2025

(54) INTELLIGENT POULTRY FARMING AUXILIARY SYSTEM AND METHOD

(71) Applicant: iCHASE Co., Ltd., Taipei City (TW)

(72) Inventors: LI-WAN HUANG, Taipei City (TW); TA-WEI CHIEN, Taipei City (TW); KUANG-FU CHANG, Taipei City (TW)

(21) Appl. No.: 19/077,054

(22) Filed: Mar. 12, 2025

Related U.S. Application Data
(60) Provisional application No. 63/564,040, filed on Mar. 12, 2024.

(51) Int. Cl. G06V 40/10; A01K 29/00; G06V 10/24; G06V 10/26; G06V 10/82

(52) U.S. Cl. CPC G06V 40/10; A01K 29/005; G06V 10/242; G06V 10/26; G06V 10/82

(57) ABSTRACT

The invention provides an intelligent poultry farming auxiliary system and method. The system includes a server and a poultry diagnostic device. The server includes a poultry image recognition artificial intelligence model. The poultry diagnostic device is connected to the server via a network and includes an image capture device and a pressure sensing device. The image capture device is configured to capture an image. When the pressure sensing device detects a pressure, the image capture device is activated to capture the image, and the poultry diagnostic device transmits the captured image to the server. The server uses the image recognition artificial intelligence model to identify a specific part of the poultry in the captured images in order to determine the poultry condition.

### CROSS-REFERENCE TO RELATED APPLICATIONS

This application claims priority of Provisional Application No. 63/564,040, filed on Mar. 12, 2024, the content of which is incorporated herein in its entirety by reference.

### FIELD OF THE INVENTION

The present invention relates to a technology for poultry farming equipment and, more particularly, to an intelligent poultry farming auxiliary system and method.

### BACKGROUND OF THE INVENTION

In traditional poultry farming, particularly in chicken farming, the process requires significant manual labor for various tedious and exhausting tasks. For example, farmers must manually catch and weigh chickens to monitor their growth and determine if adjustments to feed or environmental conditions are necessary. These tasks consume considerable time and effort and often fail to accurately reflect the health status of each chicken. Since manually catching chickens not only causes stress to the chickens, but improper stress may also cause the death of the chickens. In addition, chicken farmers must constantly observe the activities of the chickens, such as cleaning the chicken coop and adjusting the living environment of the chickens, to ensure that the chickens have sufficient activity to promote their healthy growth. These tasks require continuous manual supervision, preventing chicken farmers from leaving the farm or focusing on other work, thereby affecting task efficiency and quality of life.

On the other hand, the health status of chickens, especially disease prevention in the flock, is a very challenging task. Chicken farmers must continuously observe chicken behavior to determine if there are signs of disease. However, this manual observation relies heavily on experience and lacks comprehensive, precise health monitoring for each chicken. Moreover, chicken farmers also need to conduct night patrols to ensure the safety of the flock. This series of tasks significantly increases labor intensity of chicken farming and can easily cause excessive fatigue, which in turn affects work efficiency and the quality of chicken farming.

Furthermore, existing chicken management relies heavily on manual operations and monitoring, and it cannot achieve precise automated control. As a result, chicken farmers often experience unrelenting stress during the farming process.

### SUMMARY OF THE INVENTION

One objective of a preferred embodiment of the present invention is to provide an intelligent poultry farming auxiliary system and method, which utilizes remote imaging technology to determine the gender, weight, health status, etc., of poultry.

One objective of a preferred embodiment of the present invention is to provide an intelligent poultry farming auxiliary system and method for analyzing health conditions based on remote images of poultry excrement.

One objective of a preferred embodiment of the present invention is to provide an intelligent poultry farming auxiliary system and method for increasing the activity level of poultry.

One objective of a preferred embodiment of the present invention is to provide an intelligent poultry farming auxiliary system and method for remotely determining the average weight of poultry to keep the poultry within slaughtering standards, thereby avoiding losses during a slaughtering process.

In view of the foregoing, a preferred embodiment of the present invention provides an intelligent poultry farming auxiliary method including: providing a first image recognition artificial intelligence model; collecting a first plurality of poultry data; generating a training dataset, a validation dataset, and a testing dataset based on the first plurality of poultry data; step A: training the first image recognition artificial intelligence model through the training dataset and the validation dataset to generate a second image recognition artificial intelligence model; step B: inputting the testing dataset into the second image recognition artificial intelligence model to generate a plurality of output results; step C: collecting a second plurality of poultry data to generate the training dataset, the validation dataset, and the testing dataset when an evaluation value of the plurality of output results is below a threshold, and returning to step A until the evaluation value reaches the threshold; designating the second image recognition artificial intelligence model, in which the threshold is reached, as a third image recognition artificial intelligence model; capturing an image within a poultry farm through an image input device; and identifying a status of poultry from the image through the third image recognition artificial intelligence model.

Another preferred embodiment of the present invention provides an intelligent poultry farming auxiliary system including a server and a poultry diagnostic device. The server includes a poultry image recognition artificial intelligence model. The poultry diagnostic device is configured to connect to the server via a network, wherein the poultry diagnostic device includes an image capture device and a pressure sensing device. The image capture device is configured to capture an image. When the pressure sensing device detects a pressure, the pressure sensing device triggers the image capture device to capture the image, and the poultry diagnostic device transmits the captured image to the server. The server utilizes the poultry image recognition artificial intelligence model to identify a specific part of the poultry from the captured image, thereby determining a status of poultry.

A preferred embodiment of the present invention provides an intelligent poultry farming auxiliary system including a server and a poultry diagnostic device. The server includes a poultry image recognition artificial intelligence model. The poultry image recognition artificial intelligence model is generated by steps including: providing a first image recognition artificial intelligence model; collecting a first plurality of poultry data; generating a training dataset, a validation dataset, and a testing dataset based on the first plurality of poultry data; step A: training the first image recognition artificial intelligence model through the training dataset and the validation dataset to generate a second image recognition artificial intelligence model; step B: inputting the testing dataset into the second image recognition artificial intelligence model to generate a plurality of output results; step C: collecting a second plurality of poultry data to generate the training dataset, the validation dataset, and the testing dataset when an evaluation value of the plurality of output results is below a threshold, and returning to step A until the evaluation value reaches the threshold; and designating the second image recognition artificial intelligence model, in which the threshold is reached, as the poultry image recognition artificial intelligence model. The poultry diagnostic device is configured to connect to the server via a network and includes an image input device configured to capture an image within a poultry farm and transmit the image to the server, wherein the server utilizes the poultry image recognition artificial intelligence model to identify a status of poultry from the image.

In order to make the above and other objectives, features, and advantages of the present invention more comprehensible, preferred embodiments are described in detail below with reference to the accompanying drawings.

### BRIEF DESCRIPTION OF THE DRAWINGS

The provided drawings are intended to enable those skilled in the art to further understand the present invention and are incorporated as part of the specification of the present invention. The drawings illustrate exemplary embodiments of the present invention and are used together with the specification to describe the principles of the present invention.

FIG. 1 shows a system block diagram of an intelligent poultry farming auxiliary system according to a preferred embodiment of the present invention.

FIG. 2 shows a schematic diagram of a poultry diagnostic device of the intelligent poultry farming auxiliary system according to a preferred embodiment of the present invention.

FIG. 3 shows a schematic diagram of a poultry diagnostic device of the intelligent poultry farming auxiliary system according to a preferred embodiment of the present invention.

FIG. 4 shows a flowchart of collecting training data performed by a poultry image recognition artificial intelligence model in the intelligent poultry farming auxiliary system according to a preferred embodiment of the present invention.

FIG. 5 shows a flowchart of a training method performed by a poultry image recognition artificial intelligence model in the intelligent poultry farming auxiliary system according to a preferred embodiment of the present invention.

FIG. 6 shows a flowchart of a training method performed by a poultry image recognition artificial intelligence model in the intelligent poultry farming auxiliary system according to a preferred embodiment of the present invention.

FIG. 7 shows a flowchart of step S506 of the training method performed by the poultry image recognition artificial intelligence model in the intelligent poultry farming auxiliary system according to a preferred embodiment of the present invention.

### DETAILED DESCRIPTION

Detailed reference will be made to exemplary embodiments of the present invention, which are illustrated in the accompanying drawings. Under possible circumstances, the same reference numerals are used in the drawings and the description to refer to the same or similar components. Furthermore, the exemplary embodiments are merely some of the implementations of the design concept of the present invention, and the following examples are not intended to limit the present invention.

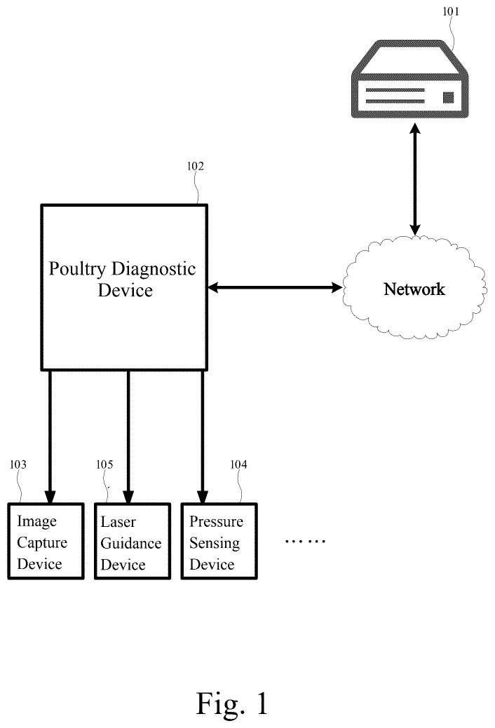

FIG. 1 shows a system block diagram of an intelligent poultry farming auxiliary system according to a preferred embodiment of the present invention. Referring to FIG. 1, an intelligent poultry farming auxiliary system includes a server 101 and a poultry diagnostic device 102. The server 101 can be deployed remotely, and the poultry diagnostic device 102 is connected to the server 101, for example, via a wired network or a wireless network. In the present embodiment, the server 101 includes a poultry image recognition artificial intelligence (AI) model. In the present embodiment, the poultry image recognition artificial intelligence model is an artificial intelligence model used for image identification.

In addition, the poultry diagnostic device 102 is deployed in a poultry farm in the present embodiment. Generally, depending on the scale of the poultry farm, multiple poultry diagnostic devices 102 can be deployed. For simplicity, only one poultry diagnostic device 102 is illustrated in the present embodiment. The poultry diagnostic device 102 includes an image capture device 103, a pressure sensing device 104, and a laser guidance device 105. In the present embodiment, the poultry diagnostic device 102 is exemplified as an expandable device. Therefore, the image capture device 103, the pressure sensing device 104, and the laser guidance device 105 are all optional components that can be added, removed, or modified according to the needs of users.

The image capture device 103 can be implemented by a digital camera or a digital camcorder for capturing images. The pressure sensing device 104, for example, is implemented by an electronic scale. In order to facilitate understanding by those skilled in the art, the poultry farm in the present embodiment is exemplified as a chicken farm, and the poultry is exemplified as chickens for illustration. those skilled in the art should understand that other poultry, such as turkeys, geese, and ducks, can also utilize the technology of the present invention, so the present invention is not limited thereto.

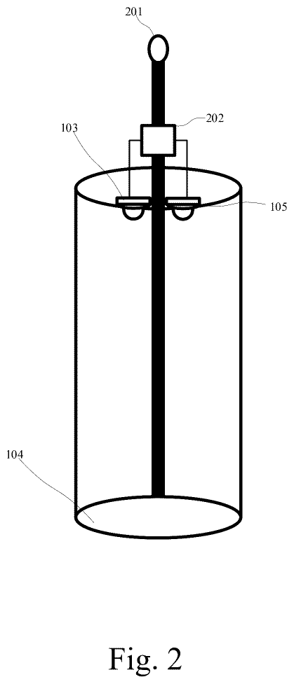

FIG. 2 shows a schematic diagram of a poultry diagnostic device of the intelligent poultry farming auxiliary system according to a preferred embodiment of the present invention. Referring to FIG. 2, in the present embodiment, the poultry diagnostic device 102 is designed in a hanging configuration with a hook 201 positioned at the top to suspend the poultry diagnostic device 102. When a chicken jumps onto the pressure sensing device 104, e.g., a weighing platform, a host device 202 receives a weight change signal from the pressure sensing device 104. Meanwhile, the host device 202 activates the image capture device 103 to capture an image of the chicken on the weighing platform. After the image is captured, the host device 202 transmits the captured image and the weight obtained from the weighing platform to the server 101 via the network. At this time, the server 101 activates the poultry image recognition artificial intelligence model to analyze the transmitted image. Generally, the poultry image recognition artificial intelligence model first identifies the comb of the chicken to determine the number of chickens and the gender of the chicken and determines whether the chicken is healthy or abnormal based on color of the comb. If any abnormality, e.g., pale comb or irregular comb shape, is determined, an alert message is immediately issued, enabling farm personnel to address the issue promptly.

The above embodiment takes the farming of broiler chickens as an example. For the farming of broiler chickens, weight and gender are crucial. Since the farming of broiler chickens typically takes about 30 to 35 days, standard slaughtering regulations require the weight of a chicken to be between 2.1 kg to 2.3 kg. If the chicken is either overweight or underweight, it can result in decreased slaughtering accuracy and potential losses. However, in the later stage of the chicken's growth, the weight of male chickens will exceed that of female chickens, and female chickens tend to be more active than male chickens. Therefore, in the later stage, female chickens are more likely than male chickens to jump or fly onto the weighing platform of the pressure sensing device 104. This situation will lead to biased statistical results of weight data and weight underestimation, which ultimately causes an average weight to be misjudged and out of spec and results in slaughtering failures (e.g., incorrect slaughtering positions leading to failed bleeding).

In the present embodiment, when the statistical data shows that the weight data of female chickens is more than that of male chickens, and the poultry image recognition artificial intelligence model of the server 101 identifies the poultry on the weighing platform of the pressure sensing device 104 as a female chicken, the server 101 notifies the host device 202 of the poultry diagnostic device 102 that the female chicken is identified, and then the host device 202 will activate the laser guidance device 105 to emit a guiding laser to make the female chicken to leave the weighing platform of the pressure sensing device 104, thereby increasing the probability of capturing the weight data of male chickens in the later stage of the chicken's growth.

In the present embodiment, the guiding laser is emitted to the outside of the weighing platform of the pressure sensing device 104 to make the female chicken to leave the weighing platform of the pressure sensing device 104. However, in practical applications, in addition to emitting the guiding laser to the outside of the weighing platform, the laser guidance device 105 can also be activated to emit the guiding laser onto the weighing platform. As a result, the guiding laser can attract chickens to jump onto the weighing platform to increasing the sampling quantity of the weight data. Emitting the guiding laser to the outside of the weighing platform can attract chickens to leave the weighing platform, preventing sampling bias. Emitting the guiding laser onto the weighing platform and to the outside at the same period not only achieves the above effects but also increases the activity level of the chicken flock.

The slaughtering weight standards and farming days in the above embodiments vary by country and may change depending on the type of poultry being raised, so the above embodiments are merely illustrative. The present invention is not limited thereto.

In addition, during the farming process, the health condition of the chickens needs to be determined through their excrement. Therefore, in the present embodiment, in order to increase the probability of capturing images of chicken excrement, when the pressure sensing device 104 detects a pressure, the host device 202 will wait for a predetermined time and then activate the laser guidance device 105 to guide the chicken off the weighing platform. Afterward, the host device 202 activates the image capture device 103 to capture an image, thereby increasing the probability of capturing images of chicken excrement. The images of chicken excrement are also transmitted via the network to the server 101, which then activates the poultry image recognition artificial intelligence model to analyze the transmitted images. If the color of the excrement is analyzed to be abnormal, such as green, red, or watery, a warning message will be issued to notify the farming staff to take prompt action.

Although the above embodiment takes the laser guidance device 105 as an example, the laser guidance device 105 is not a necessary device but an option. In another preferred embodiment, in order to increase the probability of capturing images of chicken excrement, when the pressure sensing device 104 detects a pressure, it will wait for the pressure to drop, indicating that the chicken has left the weighing platform, and then the host device 202 activates the image capture device 103 to capture an image, thereby further increasing the probability of capturing images of chicken excrement.

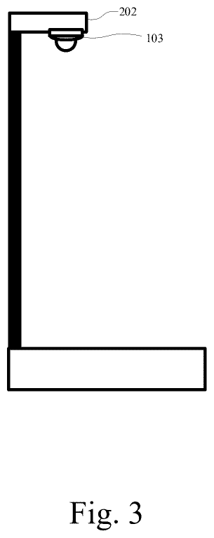

FIG. 3 shows a schematic diagram of a poultry diagnostic device of the intelligent poultry farming auxiliary system according to a preferred embodiment of the present invention. Referring to FIG. 3, in the present embodiment, the poultry diagnostic device 102 only includes the host device 202 and the image capture device 103. In the present embodiment, farmers can choose whether to use the pressure sensing device 104 based on different farming conditions. For example, taking chicken farming as an example again, however, the poultry farm is for farming egg-laying hens. Egg-laying hens are generally raised in cages, and the chickens do not move around outside, so there is no need for the pressure sensing device 104 to determine the weight of the chicken. In the present embodiment, an excrement dragging belt is provided below the cages in the egg-laying farm. When the excrement dragging belt starts to operate, the host device 202 can control the image capture device 103 to capture images of chicken excrement to obtain poultry excrement images. The poultry excrement images are transmitted via the network to the server 101, which then activates the poultry image recognition artificial intelligence model to analyze the transmitted images. If the color of the excrement is analyzed to be abnormal, such as green, red, or watery, a warning message will be issued to notify the farming staff to take prompt action.

Similarly, caged chickens are confined to a limited area, and the gender of egg-laying hens does not need to be identified. Therefore, in the present embodiment, the host device 202 will control the image capture device 103 to periodically capture images of the chickens and send the captured images to the server 101. The server 101 will then activate the poultry image recognition artificial intelligence model to analyze the transmitted images. If the shape or color of the comb is analyzed to be abnormal, a warning message will be issued to notify the farming staff to take prompt action.

Although the above embodiment takes comb-based gender identification as an example, those skilled in the art should understand that different gender identification methods may be adopted based on different poultry species. For example, the gender of cherry valley ducks is identified by the curling degree of their tail feathers. Similarly, a male cherry valley duck is generally heavier than a female cherry valley duck in the later stage of the duck's growth. Therefore, the present invention is not limited to the above embodiments.

In the above embodiments, although the poultry image recognition artificial intelligence model is configured to recognize images for determining gender, comb health status, excrement status, etc., those skilled in the art should understand that the weight of poultry actually has a relative relationship with its appearance. Therefore, the poultry image recognition artificial intelligence model can also predict or estimate the weight of poultry from the images. Therefore, the present invention is not limited to the above applications.

The above embodiments illustrate the operation of the intelligent poultry farming auxiliary system. The following embodiments will describe a method for generating the poultry image recognition artificial intelligence model.

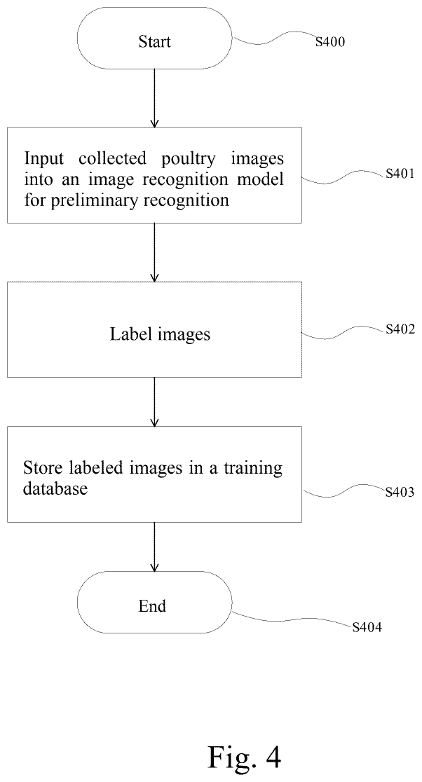

FIG. 4 shows a flowchart of collecting training data performed by a poultry image recognition artificial intelligence model in the intelligent poultry farming auxiliary system according to a preferred embodiment of the present invention. Referring to FIG. 4, the steps for collecting training data include:

Step S400: Start.

Step S401: Collect poultry images and input the collected poultry images into an image recognition model for preliminary recognition. Generally, the collecting step can be continuously performed through the image capture device 103 of the poultry diagnostic device 102 to collect images. The preliminary recognition will mark which parts are chickens and positions of the chickens in the images.

Step S402: Label the images. Specifically, the comb, gender, normal health status and abnormal health status are labeled based on the chickens and their positions in the image. In the present embodiment, the labeling step can be performed by a previously trained image recognition model to label features. As a result, the speed of labeling features is accelerated.

Step S403: Store the labeled images in a training database.

Step S404: End.

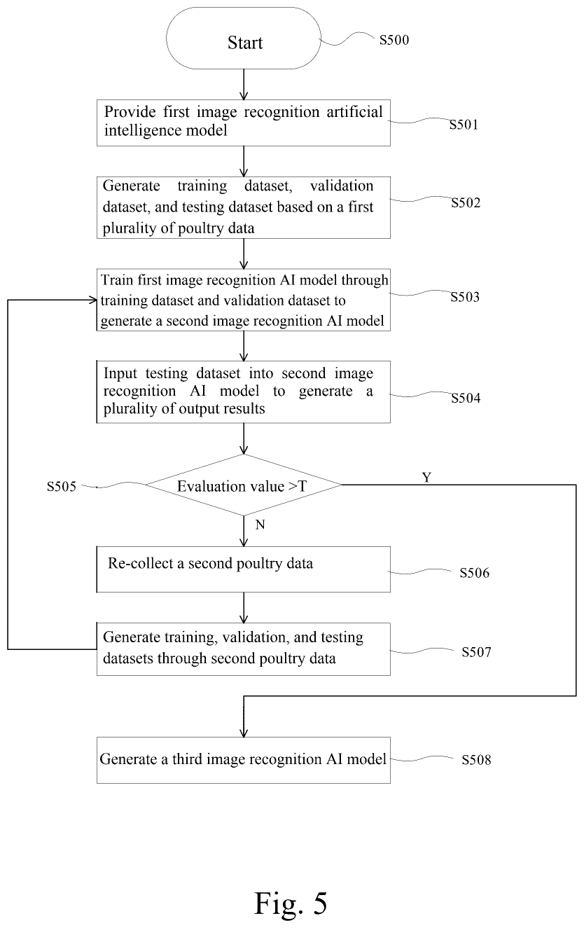

FIG. 5 shows a flowchart of a training method performed by a poultry image recognition artificial intelligence model in the intelligent poultry farming auxiliary system according to a preferred embodiment of the present invention. Referring to FIG. 5, the training method performed by the poultry image recognition artificial intelligence model includes the following steps:

Step S500: Start.

Step S501: Provide a first image recognition artificial intelligence (AI) model. This first image recognition artificial intelligence model is, for example, an initial image recognition artificial intelligence model.

Step S502: Generate a training dataset, a validation dataset, and a testing dataset based on a first plurality of poultry data. In the present embodiment, not all the poultry image data collected in FIG. 4 will be used; instead, only a portion of the poultry image data will be extracted, and image data collection in FIG. 4 will continue. The extracted poultry data will generate the training dataset, the validation dataset, and the testing dataset. Afterwards, in order to enhance data diversity and improve model stability, data augmentation, such as adjustment to brightness, adjustment to segmentation, and adjustment to rotation, will first be performed to generate more training images based on the collected images in the same database. Accordingly, with diversified training data, the model can better adapt to real-world variations, such as different angles, sizes, and colors, thereby improving the stability and accuracy of the model. In the present embodiment, these training images are divided into the training dataset, validation dataset, and testing dataset in a 4:1:1 ratio.

Step S503: Train the first image recognition artificial intelligence model through the training dataset and the validation dataset to generate a second image recognition artificial intelligence (AI) model. In the present embodiment, the first image recognition artificial intelligence model employs, for example, a convolutional neural network (CNN). Since convolutional operation is more effective at extracting local features (such as edges, colors, and textures), it is more suitable for image processing, image feature extraction, etc. However, the present invention is not limited to other implementations, such as recurrent neural networks (RNN) and long short-term memory (LSTM).

During a training process, images in the training dataset and images in the validation dataset each have the labeled features, which provides true answers, that is, the category and location of the poultry in each image. These labeled features help the first image recognition artificial intelligence model understand the correct answers and enable it to learn effectively. This first image recognition artificial intelligence model adjusts its internal parameters (such as weights) by repeating the training process. During the training process, the first image recognition artificial intelligence model generates a prediction result, which is compared to a labeled result, and is adjusted based on the difference in error identified in the comparison.

After completing the training with the training dataset, the training process proceeds to the next step, which is to perform a performance evaluation with the validation dataset. During the performance evaluation, a performance evaluation metric, such as mean average precision (e.g., mAP50) can also be used for the evaluation. The performance evaluation will return images, which fall below a threshold in the validation dataset, to the first image recognition artificial intelligence model for repeating the training process until a convergent result is achieved to obtain a second image recognition artificial intelligence model.

Step S504: Input the testing dataset into the second image recognition artificial intelligence model to generate a plurality of output results.

Step S505: Determine whether an evaluation value of the plurality of output results is below an evaluation threshold T. If the evaluation value is below the evaluation threshold T, proceed to Step S506. In the present embodiment, the evaluation threshold T uses the mean average precision at IoU=0.5 (mAP50) as the evaluation metric in the field of object detection. It is used to measure the accuracy of the model in recognizing objects in image detection tasks. Those skilled in the art should understand that the method for calculating the threshold for training results can vary depending on different applications. Therefore, the present invention is not limited to mAP50 in the present embodiment.

Step S506: Re-collect a second plurality of poultry data. When the evaluation value in Step S505 fails to reach the threshold, it indicates insufficient training or inadequate data volume. Meanwhile, the image data collected in FIG. 4 is extracted again. Since the image capture device 103 of the poultry diagnostic device 102 is deployed in the poultry farm and continuously collects data, it can obtain a second plurality of poultry data different from a first plurality of poultry data previously obtained.

Step S507: Generate the training dataset, the validation dataset, and the testing dataset through the second plurality of poultry data. After labeling and performing data augmentation on the training dataset, validation dataset, and testing dataset, return to Step S503 to retrain the second image recognition artificial intelligence model until the evaluation value reaches the threshold.

Step S508: Generate a third image recognition artificial intelligence (AI) model. When the evaluation value in Step S505 is passed, or when the evaluation value reaches the threshold but cannot be further improved, it indicates that the training process has reached a convergence stage. Meanwhile, further repeated training yields no additional improvements, signifying the completion of the training process. As a result, the third image recognition artificial intelligence model finally generated serves as the poultry image recognition artificial intelligence model.

From the above embodiment, it can be seen that the training dataset is the dataset used to train the image recognition artificial intelligence model. The image recognition artificial intelligence model learns from the data and adjusting its internal parameters to improve prediction accuracy. From the perspective of general learning, it can be likened to a textbook. The validation dataset is used to evaluate the performance of the image recognition artificial intelligence model during the training process and helps in adjusting parameters. It does not participate in the training of the image recognition artificial intelligence model but is used to assess the learning progress of the image recognition artificial intelligence model. From the perspective of general learning, it can be likened to exercises with answers. The testing dataset is the dataset used to evaluate the performance of the image recognition artificial intelligence model. The testing dataset is entirely unused during the training and validation of the image recognition artificial intelligence model and serves to assess the performance of the image recognition artificial intelligence model on new data. From the perspective of general learning, it can be likened to exercises without answers to evaluate learning outcomes. Therefore, in the above embodiment, it can be seen that the training dataset and validation dataset include images with labeled features while the testing dataset includes images without labeled features.

In the above embodiment, for the images in the testing dataset that were incorrectly identified, they can be relabeled and reused as training dataset, and the original second image recognition artificial intelligence model is then retrained to generate a third image recognition artificial intelligence model. Therefore, with the repeated training process described above, the third image recognition artificial intelligence model, i.e., the poultry image recognition artificial intelligence model, is further strengthened.

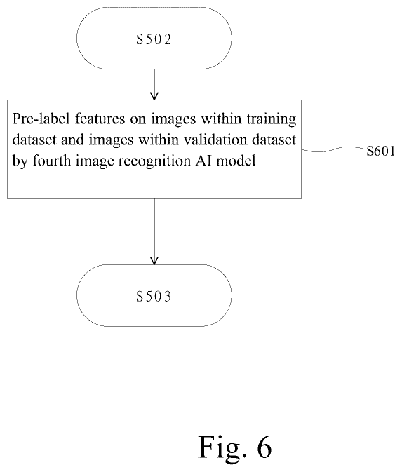

FIG. 6 shows a flowchart of a training method performed by a poultry image recognition artificial intelligence model in the intelligent poultry farming auxiliary system according to a preferred embodiment of the present invention. Referring to FIG. 6, the training method performed by the poultry image recognition artificial intelligence model, before step S503, further includes the following steps:

Step S601: Pre-label features on images within the training dataset and images within the validation dataset by a fourth image recognition artificial intelligence (AI) model. In the present embodiment, the fourth image recognition artificial intelligence model may be a previous version of the poultry image recognition artificial intelligence model. As a result, the learning outcomes of the previous version of the poultry image recognition artificial intelligence model can be transferred to the subsequently trained third image recognition artificial intelligence model, facilitating faster training of the image recognition artificial intelligence model.

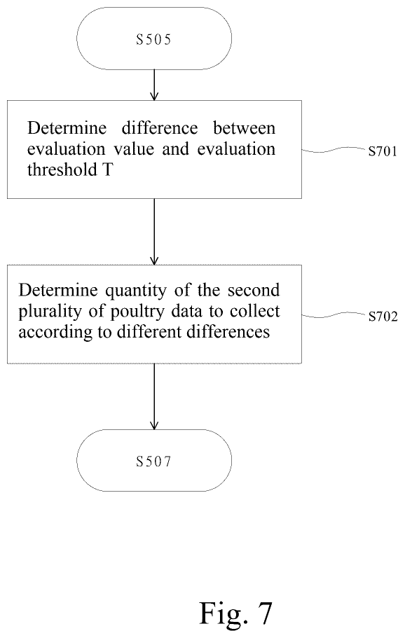

FIG. 7 shows a flowchart of step S506 of the training method performed by the poultry image recognition artificial intelligence model in the intelligent poultry farming auxiliary system according to a preferred embodiment of the present invention. Referring to FIG. 7, step S506 of the training method performed by the poultry image recognition artificial intelligence model further includes the following steps:

Step S701: Determine the difference between the evaluation value and the evaluation threshold T.

Step S702: Determine the quantity of the second plurality of poultry data to collect according to different differences. For example, if the difference between the evaluation value and the evaluation threshold T is within 5%, the quantity of the second plurality of poultry data may be half that of the first plurality of poultry data. If the difference is between 5% and 10%, the quantity of the second plurality of poultry data may equal that of the first plurality of poultry data. If the difference exceeds 10%, the quantity of the second plurality of poultry data may be twice that of the first plurality of poultry data. As a result, the number of training cycles and the computational load during training can be reduced.

In summary, the preferred embodiment of the present invention combines a poultry image recognition artificial intelligence model with data augmentation technology, which not only effectively improves the accuracy of automated monitoring in poultry farming but also significantly enhances the efficiency of farming management. The present method involves classifying multiple poultry datasets, repeatedly training and adjusting the model by using training dataset, validation dataset, and testing dataset to ensure high-precision recognition capabilities in various scenarios. When erroneous results appear in the testing dataset, the model can employ data augmentation technology to generate more training samples, thereby optimizing its performance. The present method not only improves the accuracy of recognition results but also enables farming staff to achieve real-time monitoring of poultry growth status, leading to refined management, reduced labor costs, increased production efficiency, and other advantages and making it highly valuable for the poultry farming industry.

The specific embodiments described in the detailed description of the preferred embodiments are provided solely to facilitate the understanding of the technical content of the present invention and should not be construed as narrowly limiting the present invention to the above embodiments. Any modifications made without departing from the spirit of the present invention and the scope of the appended claims fall within the scope of the present invention. Therefore, the protection scope of the present invention shall be determined by the appended claims.

What is claimed is:

1. An intelligent poultry farming auxiliary method, comprising:
providing a first image recognition artificial intelligence model;
collecting a first plurality of poultry data;
generating a training dataset, a validation dataset, and a testing dataset based on the first plurality of poultry data;
step A: training the first image recognition artificial intelligence model through the training dataset and the validation dataset to generate a second image recognition artificial intelligence model;
step B: inputting the testing dataset into the second image recognition artificial intelligence model to generate a plurality of output results;
step C: collecting a second plurality of poultry data to generate the training dataset, the validation dataset, and the testing dataset when an evaluation value of the plurality of output results is below a threshold, and returning to step A until the evaluation value reaches the threshold;
designating the second image recognition artificial intelligence model, in which the threshold is reached, as a third image recognition artificial intelligence model;
capturing an image within a poultry farm through an image input device, wherein there are a plurality of poultry in the poultry farm; and
identifying a status of one of the plurality of poultry from the image through the third image recognition artificial intelligence model.

2. The method of claim 1, wherein step A further comprises one of steps:
adjusting brightness of images in the training dataset and images in the validation dataset to increase a quantity of the images in the training dataset and images in the validation dataset;
adjusting segmentation of images in the training dataset and images in the validation dataset to increase a quantity of the images in the training dataset and images in the validation dataset; and
adjusting rotation of images in the training dataset and images in the validation dataset to increase a quantity of the images in the training dataset and images in the validation dataset.

3. The method of claim 1, wherein capturing the image within the poultry farm further comprises:
capturing the image when a pressure sensing device detects a pressure.

4. The method of claim 1, wherein identifying the status of one of the plurality of poultry from the image further comprises:
determining a gender of the one poultry through a comb of the one poultry.

5. The method of claim 1, wherein identifying the status of one of the plurality of poultry from the image further comprises:
determine a health status of the one poultry through a comb of the one poultry.

6. The method of claim 1, wherein when at least two poultry are present in the image, the method further comprises:
identifying a comb of each of the at least two poultry to obtain a comb image; and
extracting the comb from the comb image and comparing the comb with the image to respectively locate a corresponding poultry.

7. The method of claim 1, wherein capturing the image within the poultry farm further comprises:
capturing a second image to obtain a poultry excrement image after a pressure sensing device detects a pressure and waits for the pressure to drop.

8. The method of claim 7, wherein capturing the second image to obtain the poultry excrement image further comprises:
activating a laser guidance device to emit a guiding laser to guide at least one poultry to leave the pressure sensing device when the at least one poultry stays on the pressure sensing device for a predetermined time.

9. The method of claim 1, wherein capturing the image within the poultry farm further comprises:
providing an excrement dragging belt within the poultry farm; and
capturing the image to obtain a poultry excrement image when the excrement dragging belt starts to operate.

10. The method of claim 1, wherein capturing the image within the poultry farm further comprises:
capturing the image when a pressure sensing device detects a weight, identifying a gender of the one poultry from the image through the third image recognition artificial intelligence model, and storing the gender and the weight corresponding to the gender; and
guiding, by a laser guidance device, at least one first poultry with a first gender to leave the pressure sensing device to increase a sampling probability of at least one second poultry with a second gender when a first number of poultry with the first gender exceeds a second number of poultry with the second gender and when the at least one first poultry staying on the pressure sensing device is identified as the first gender.

11. The method of claim 1, wherein images within the training dataset and images within the validation dataset include labeled features.

12. The method of claim 11, further comprising:
pre-labeling features on images within the training dataset and images within the validation dataset by a fourth image recognition artificial intelligence model.

13. An intelligent poultry farming auxiliary system, comprising:
a server including a poultry image recognition artificial intelligence model; and
a poultry diagnostic device configured to connect to the server via a network, wherein the poultry diagnostic device includes:
an image capture device configured to capture an image of at least one poultry; and
a pressure sensing device;
wherein when the pressure sensing device detects a pressure, the pressure sensing device triggers the image capture device to capture the image, and the poultry diagnostic device transmits the captured image to the server; and
wherein the server utilizes the poultry image recognition artificial intelligence model to identify a specific part of the at least one poultry from the captured image, thereby determining a status of the at least one poultry.

14. The system of claim 13, wherein the poultry diagnostic device is further configured to:
transmit a pressure data obtained by the pressure sensing device and the image to the server,
wherein the server is configured to record poultry data based on the pressure data and based on a number and a gender of the at least one poultry identified from the image and determine a growth status according to statistical results from the recorded poultry data.

15. The system of claim 14, wherein the poultry diagnostic device further comprises:
a laser guidance device configured to emit a guiding laser, wherein:
the laser guidance device is activated to guide the at least one poultry on the pressure sensing device to leave the pressure sensing device when the recorded poultry data shows that a first number of poultry with a first gender is greater than a second number of poultry with a second gender and when the at least one poultry in the image is determined to be the first gender.

16. The system of claim 13, wherein the poultry diagnostic device further comprises:
a laser guidance device configured to emit a guiding laser, wherein the poultry diagnostic device is further configured to:
activate the laser guidance device to guide the at least one poultry on the pressure sensing device to leave the pressure sensing device when the pressure sensing device detects the pressure;
activate the image capture device to capture the image to obtain an excrement image of the at least one poultry; and
transmit the excrement image to the server;
wherein the poultry image recognition artificial intelligence model determines a health status of the at least one poultry based on the excrement image.

17. The system of claim 13, wherein a second image is captured to obtain a poultry excrement image after the pressure sensing device detects the pressure and waits for the pressure to drop.

18. The system of claim 13, wherein the poultry image recognition artificial intelligence model further performs steps comprising:
providing a first image recognition artificial intelligence model;
generating a training dataset, a validation dataset, and a testing dataset based on a first plurality of poultry data;
step A: training the first image recognition artificial intelligence model through the training dataset and the validation dataset to generate a second image recognition artificial intelligence model;
step B: inputting the testing dataset into the second image recognition artificial intelligence model to generate a plurality of output results;
step C: collecting a second plurality of poultry data to generate the training dataset, the validation dataset, and the testing dataset when an evaluation value of the plurality of output results is below a threshold, and returning to step A until the evaluation value reaches the threshold; and
designating the second image recognition artificial intelligence model, in which the threshold is reached, as the poultry image recognition artificial intelligence model.

19. The system of claim 18, wherein step A further comprises one of steps:
adjusting brightness of images in the training dataset and images in the validation dataset to increase a quantity of the images in the training dataset and images in the validation dataset;
adjusting segmentation of images in the training dataset and images in the validation dataset to increase a quantity of the images in the training dataset and images in the validation dataset; and
adjusting rotation of images in the training dataset and images in the validation dataset to increase a quantity of the images in the training dataset and images in the validation dataset.

20. The system of claim 19, further comprising:
pre-labeling, by a fourth image recognition artificial intelligence model, features on images within the training dataset and images within the validation dataset to train the poultry image recognition artificial intelligence model.

21. An intelligent poultry farming auxiliary system, comprising:
a server including a poultry image recognition artificial intelligence model, wherein the poultry image recognition artificial intelligence model performs steps comprising:
providing a first image recognition artificial intelligence model;
generating a training dataset, a validation dataset, and a testing dataset based on a first plurality of poultry data;
step A: training the first image recognition artificial intelligence model through the training dataset and the validation dataset to generate a second image recognition artificial intelligence model;
step B: inputting the testing dataset into the second image recognition artificial intelligence model to generate a plurality of output results;
step C: collecting a second plurality of poultry data to generate the training dataset, the validation dataset, and the testing dataset when an evaluation value of the plurality of output results is below a threshold, and returning to step A until the evaluation value reaches the threshold;
designating the second image recognition artificial intelligence model, in which the threshold is reached, as the poultry image recognition artificial intelligence model; and
a poultry diagnostic device connected to the server via a network and including an image input device configured to capture an image within a poultry farm and transmit the image to the server, wherein the server utilizes the poultry image recognition artificial intelligence model to identify a status of poultry from the image.

22. The system of claim 21, wherein step A further comprises one of steps:
adjusting brightness of images in the training dataset and images in the validation dataset to increase a quantity of the images in the training dataset and images in the validation dataset;
adjusting segmentation of images in the training dataset and images in the validation dataset to increase a quantity of the images in the training dataset and images in the validation dataset; and
adjusting rotation of images in the training dataset and images in the validation dataset to increase a quantity of the images in the training dataset and images in the validation dataset.

23. The system of claim 21, wherein the poultry image recognition artificial intelligence model further performs steps comprising:
pre-labeling, by a fourth image recognition artificial intelligence model, features on images within the training dataset and images within the validation dataset to train the poultry image recognition artificial intelligence model.

24. The system of claim 21, wherein the poultry diagnostic device further comprises:
a pressure sensing device, wherein:
when the pressure sensing device detects the pressure, the pressure sensing device triggers the image capturing device to capture the image;
wherein the poultry diagnostic device transmits a pressure data obtained by the pressure sensing device and the image to the server; and
wherein the server is configured to record poultry data based on the pressure data and based on a number and a gender of the at least one poultry identified from the image and determine a growth status according to statistical results from the recorded poultry data.

25. The system of claim 21, wherein the poultry diagnostic device further comprises:
a laser guidance device configured to emit a guiding laser, wherein:
the laser guidance device is activated to guide the at least one poultry on the pressure sensing device to leave the pressure sensing device when the recorded poultry data shows that a first number of poultry with a first gender is greater than a second number of poultry with a second gender and when the at least one poultry in the image is determined to be the first gender.

26. The system of claim 21, wherein the poultry diagnostic device further comprises:
a laser guidance device configured to emit a guiding laser, wherein the poultry diagnostic device is further configured to:
activate the laser guidance device to guide the at least one poultry on the pressure sensing device to leave the pressure sensing device when the pressure sensing device detects the pressure;
activate the image capture device to capture the image to obtain an excrement image of the at least one poultry; and
transmit the excrement image to the server;
wherein the poultry image recognition artificial intelligence model determines a health status of the at least one poultry based on the excrement image.

27. The system of claim 21, wherein a second image is captured to obtain a poultry excrement image after the pressure sensing device detects the pressure and waits for the pressure to drop.
